(19) United States
(12) Patent Application Publication
(10) Pub. No.: US 2012/0071826 A1
(43) Pub. Date: Mar. 22, 2012

(54) SAFE SELF-DESTRUCTION SYRINGE WITH PUNCTURE PROOF PROTECTIVE SLEEVE

(75) Inventors: Zhong Feng, Jiangsu Province (CN); Mike Feng, Jiangsu Province (CN)

(73) Assignee: WUXI YUSHOU MEDICAL APPLICANCE LTD., Wuxi, Jiangsu (CN)

(21) Appl. No.: 13/138,613

(22) PCT Filed: Oct. 8, 2010
(86) PCT No.: PCT/CN2010/001553

§ 371 Date: Nov. 28, 2011

(30) Foreign Application Priority Data
Oct. 9, 2009 (CN) 200910205361.9

(51) Int. Cl. A61M 5/50

(52) U.S. Cl. 604/110

(57) ABSTRACT

A safety self-destruction syringe with puncture proof protective sleeve consists of a needlepoint protective sleeve (1), a needlepoint (2), a needle base (3), a puncture proof protective sleeve (4), an adapter member (5), a locking bayonet (6), a liquid pushing cylinder sleeve (7), a core rod (8), and a sealing plug (9). The adapter member (5) is arranged on the top end of the puncture proof protective sleeve (4). The locking baonet (6) is arranged on the puncture proof protective sleeve (4) by bonding or welding. The thread of the Luer connector (10) at the top end of the liquid pushing cylinder sleeve (7) is tightly matched with or engaged with lugs (16) of the adapter member (5). The locking bayonet (6) has elastic hook pieces (18) for locking the adapter member (5).

### BACKGROUND OF THE PRESENT INVENTION

1. Field of Invention

The present invention relates to a syringe, and more particularly to a syringe comprising a puncture proof protective sleeve for preventing a user of the syringe to be accidentally hurt by the syringe.

2. Description of Related Arts

Nowadays, almost all medical personnel utilize conventional syringes for injections. A disadvantage of convention syringes is that the needle is not shielded and exposed to an exterior of the syringe so that medical or cleaning staff may easily be hurt by the needles. Because of this problem, syringes having some sorts of safety devices have been developed to avoid used needles to accidentally touch other people. Such safety devices include self-destruction type syringes and syringes with retractable needles. Chinese patent number ZL200410092337.6 and ZL 20040121924.9 disclose syringes with movable caps and represent improvements over the syringes disclosed in U.S. Pat. No. 5,338,310. For these conventional syringes however, the cap cannot be detached from the syringe body. This makes the overall size of the syringes become very bulky, and greatly increases the handling costs of used syringes. Moreover, when the cap is detached from the syringe body, the needle is still exposed to an exterior thereof, and the syringe may easily be reused. Chinese patent number ZL200410092337.6 discloses a syringe in which the protective cap can be detached from the syringe body. This design decreases the handling costs for used syringes, yet it does not prevent the syringe from being easily reused. Chinese patent number ZL2004201219249 and ZL 200410092337.6 discloses another type of syringe in which the various components of the syringes are designed not to follow international standards so as to make reuse of the syringe body much more difficult. However, this type of design makes the hands of the medical staff uncomfortable because of many protrusions formed on an outer side of the syringe body.

### SUMMARY OF THE PRESENT INVENTION

The invention is advantageous in that it provides a syringe comprising a puncture proof protective sleeve for preventing a user of the syringe to be accidentally hurt by the syringe. Moreover, the present invention improves upon Chinese patent number ZL200410092337.6 and ZL200420121924.9, and does not impart uncomfortable feeling to the hands of the relevant medical staff. Moreover, the needle of the present invention is securely retained within a protective sleeve for enhancing a safety feature thereof.

Accordingly, in order to solve the above technical problem, the present invention provide a syringe, comprising a needle cap 1, a needle 2, a needle seat 3, a protective sleeve 4, a coupling device 5, a locking member 6, a syringe body 7, a plunger shaft 8, and a plunger head 9.

In particular, the needle 2 is affixed to and extended from the needle seat 3, wherein the needle seat is detachably connected to the coupling device 5, which is provided at a top end portion of the protective sleeve 4, while the locking member 6 is received in another end portion of the protective sleeve 4, wherein the locking member is connected with a lower end surface 12 through conventional adhesive agent or welding technology by means of a welding wire 17, wherein the plunger head 9 is provided at a top end portion of the plunger shaft 8, which are movably received within the syringe body, wherein the syringe body, the plunger shaft, and the plunger head are all operatively received in the protective sleeve, wherein the syringe body 7 has a luer taper 10 for connecting with the coupling device through threading mechanism, wherein the coupling device has a circular cross section defining an inner cavity 15, and a plurality of coupling members 16 spacedly extended on the coupling device, wherein the coupling members 16 are arranged to couple with the luer taper 10 of the syringe body, wherein the syringe further comprises a resilient flanges 18 operatively connected with the locking device 6 which interlocks with the needle 2, the needle seat 3 and the coupling device 5 by operation of a sliding action of the flanges 18 in a backward direction.

According to the syringe of the present invention, the syringe body further comprises an interlocking ring 23 extended from the luer tape 10, wherein the coupling device 5 further has an interlocking slot 24 formed thereon, wherein the interlocking ring 23 is arranged to couple with the interlocking slot 24 so as to enhance a coupling performance between the syringe body and the coupling device.

According to the syringe of the present invention, the plunger shaft 8 and the plunger head 9 are integrally molded together for receiving in the syringe body, thereby the coupling device 5 and the luer taper 10 of the syringe body 7 are connected in such a manner that a gap is defined between the coupling device 5 and the luer taper 10 of the syringe body 7 through which a residue fluid retained inside the syringe body is restricted for meeting an international standard, wherein the plunger head 9 has a conical shape which is adapted to receive in the luer taper for ejecting a predetermined of fluid from the syringe body.

According to the syringe of the present invention, the coupling device has a circular cross section defining an inner cavity 15, and a plurality of coupling members 16 spacedly extended on the coupling device, wherein the coupling members 16 are arranged to couple with the luer taper 10 of the syringe body.

According to the syringe of the present invention, said protective sleeve 4 has an external smooth surface.

Solution 1: since Chinese patent numbers ZL200410092337.6 and ZL 200420121924.9 cause the hands of the relevant medical staff uncomfortable because of the existence of many protrusions on the syringe, the outer surface of the syringe of the present invention will be kept as smooth as possible.

100111 Solution 2: since the syringe disclosed in Chinese patent number ZL200410092337.6 need sanitization by high temperature, the syringe body suffers from slight deformation due to huge temperature variation. As such, the connection between the needle and the syringe body may become loose. When the syringes are transported from one place to another, the loose needle may detach from the syringe body and the needle may hurt people who are responsible for handling the syringes. The present invention requires that the syringe body 7 comprises a luer taper for connecting with the coupling device through threading mechanism. On the other hand, the coupling device has a circular cross section defining an inner cavity, and a plurality of coupling members spacedly extended on the coupling device, wherein the coupling members are arranged to couple with the luer taper of the syringe body. This arrangement ensures tight and secure connection between the syringe body and the needle irrespective of weather conditions.

### BRIEF DESCRIPTION OF THE DRAWINGS

FIG. 1 is a schematic diagram of a syringe according to a preferred embodiment of the present invention.

FIG. 2 is an exploded side view of a syringe according to the above preferred embodiment of the present invention.

FIG. 3 is a side view of the coupling device of the syringe according to the above preferred embodiment of the present invention.

FIG. 4 is a schematic diagram of the locking member of the syringe according to the above preferred embodiment of the present invention.

FIG. 5 is a schematic diagram of the protective sleeve of the syringe according to the above preferred embodiment of the present invention.

FIG. 6 is a schematic diagram of the syringe according to the above preferred embodiment of the present invention, illustrating that the syringe is used for an injection.

FIG. 7 is a zoom in view (6A) of FIG. 6 according to the above preferred embodiment of the present invention.

FIG. 8 is another schematic diagram of the syringe according to the above preferred embodiment of the present invention.

FIG. 9 is a first alternative mode of the syringe according to the above preferred embodiment of the present invention.

FIG. 10 is a schematic diagram of the plunger shaft and the plunger head according to the above preferred embodiment of the present invention.

FIG. 11 is a second alternative mode of the syringe according to the above preferred embodiment of the present invention.

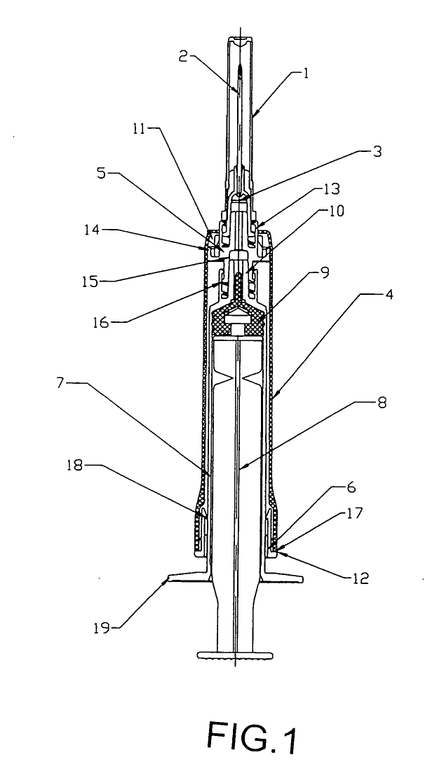

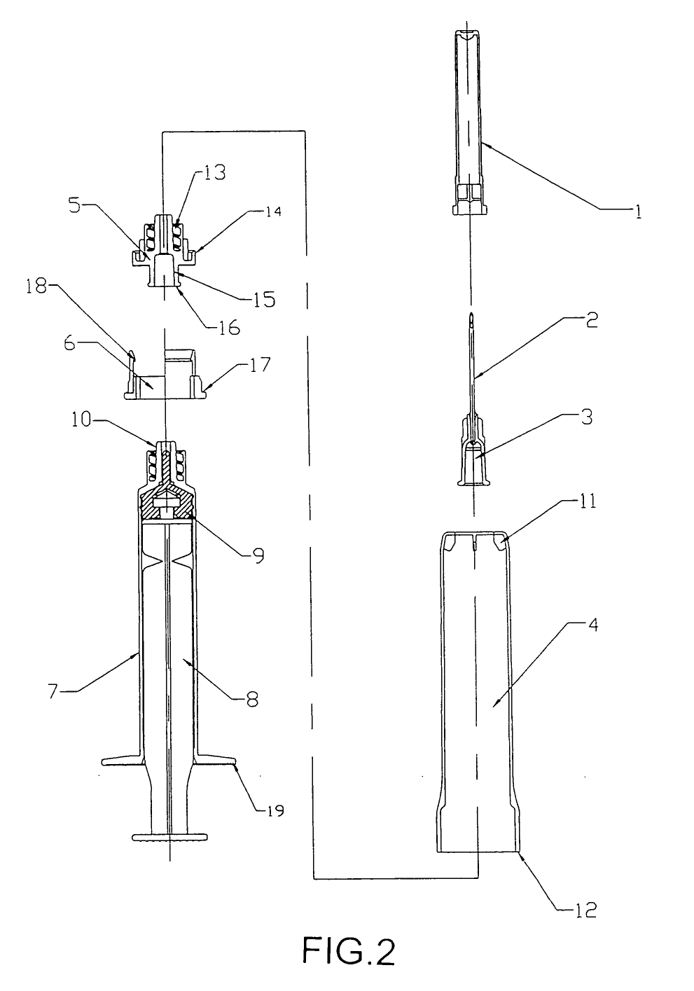

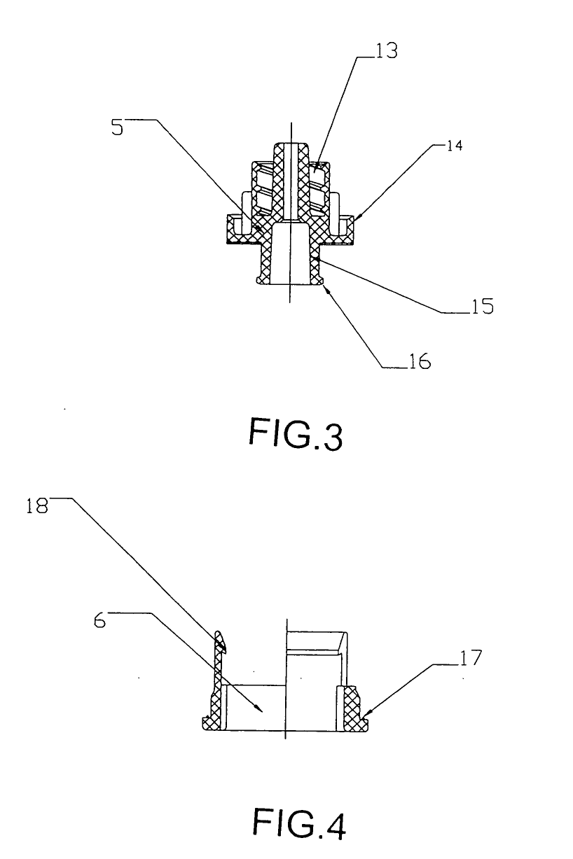

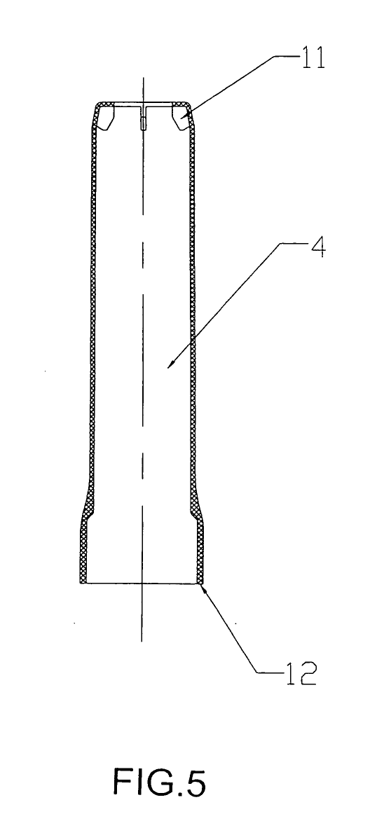

1—needle cap, 2—needle, 3—needle seat, 4—protective sleeve, 5—coupling device, 6—locking member, 7—syringe body, 8—plunger shaft, 9—plunger head, 10—luer taper, 11—blocking member, 12—lower end surface, 13—connection head, 14—protruding member, 15—inner cavity, 16—coupling member, 17—welding wire, 18—resilient flanges, 19—handling sleeve, 20—rotatable needle seat, 21—plunger shaft body, 22—another coupling device, 23—interlocking ring, 24—interlocking slot.

### DETAILED DESCRIPTION OF THE PREFERRED EMBODIMENT

The detailed description of the preferred embodiment of the present invention is as follows:

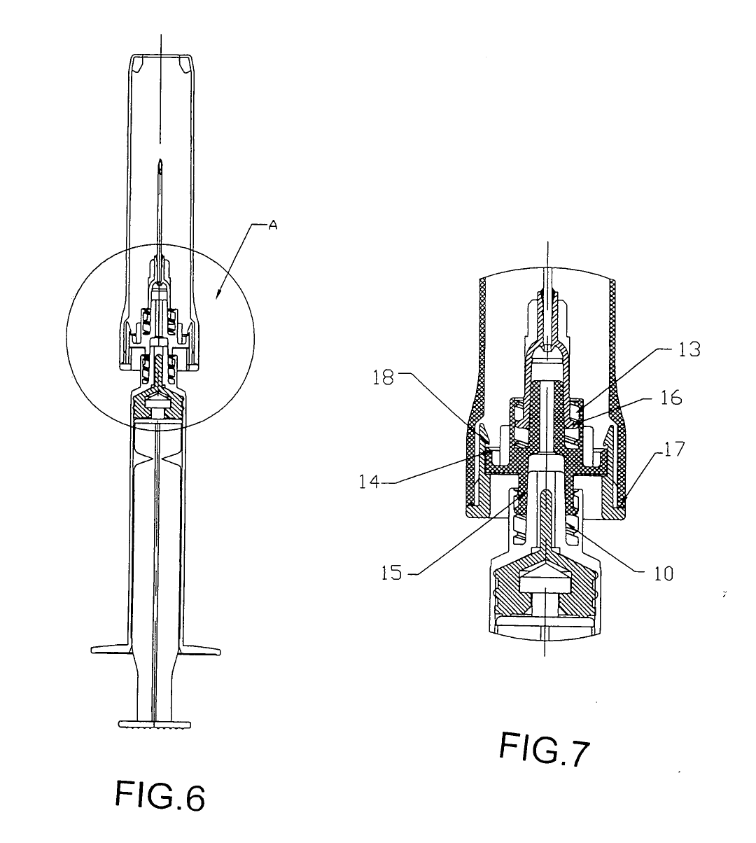

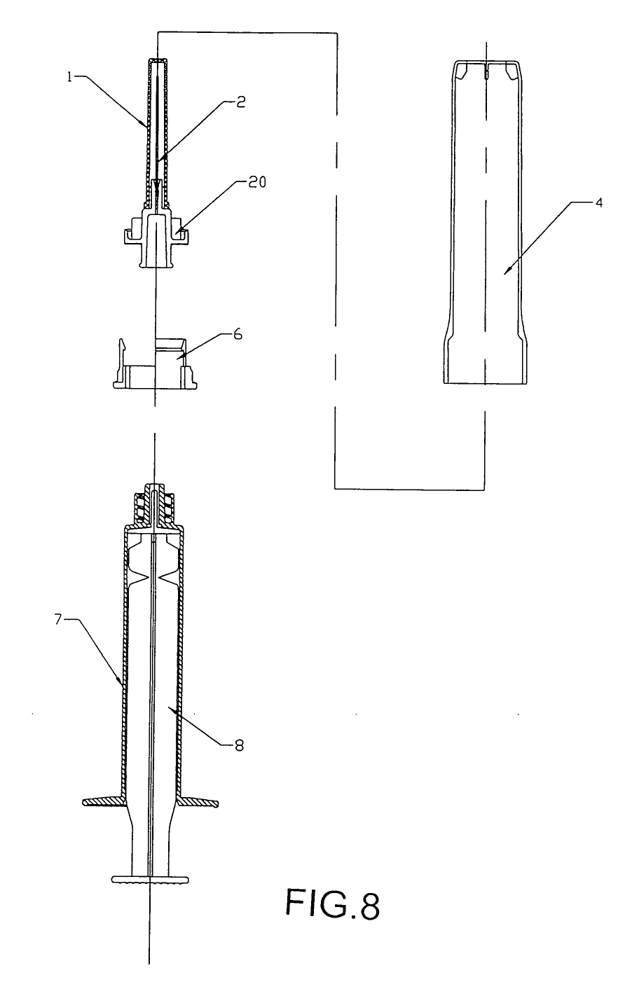

Referring to the drawings, the syringe according to the preferred embodiment of the present invention comprises a needle cap 1, a needle 2, a needle seat 3, a protective sleeve 4, a coupling device 5, a locking member 6, a syringe body 7, a plunger shaft 8, and a plunger head 9. In this preferred embodiment, the coupling device 5 is provided at a top end portion of the protective sleeve 4, while the locking member 6 is received in another end portion of the protective sleeve 4, wherein the locking member 6 is connected with a lower end surface 12 through conventional adhesive agent or welding technology by means of a welding wire 17 or electrode. Moreover, the plunger head 9 is provided at a top end portion of the plunger shaft 8, which are movably received within the syringe body 7. The syringe body 7, the plunger shaft 8, and the plunger head 9 are all operatively received in the protective sleeve 4. The syringe body 7 has a luer taper 10 for connecting with the coupling device 5 through threading mechanism. On the other hand, the coupling device 5 has a circular cross section defining an inner cavity 15, and a plurality of coupling members 16 spacedly extended on the coupling device 5, wherein the coupling members 16 are arranged to couple with the luer taper 10 of the syringe body 7. Furthermore, the needle 2 is extended from the needle seat 3 while the needle cap 1 is detachably affixed on the needle seat 3 to prevent the needle 2 from being exposed to ambient environment. The coupling device 5 further has a connection head 13 wherein the needle seat 3 is detachably attached on the connecting head 13. When the syringe is in clinical use, medical staff may, depending on medical or clinical needs, attach different needle 2 with needle seat 3 an the needle cap 1 onto the coupling device 5. For an injection cycle, a user may first hold a handling sleeve 19 and pulling the plunger shaft 8 backwardly for filling a predetermined medicine into the syringe body 7. When the injection process is finished, the user may slide the protective sleeve 4 toward the needle seat 3 until the locking device 6 is interlocked with the coupling device 5 by means of a resilient flanges 18 (see FIG. 6). It is worth mentioning that the coupling device 5 can be easily detached from the syringe body 7 by rotating the coupling device 5 in a predetermined direction. The syringe disclosed in the present invention improves upon conventional syringes and has a simple structure which facilitates safe and efficient injections. The syringe of the present invention also complies with major international safety standards.

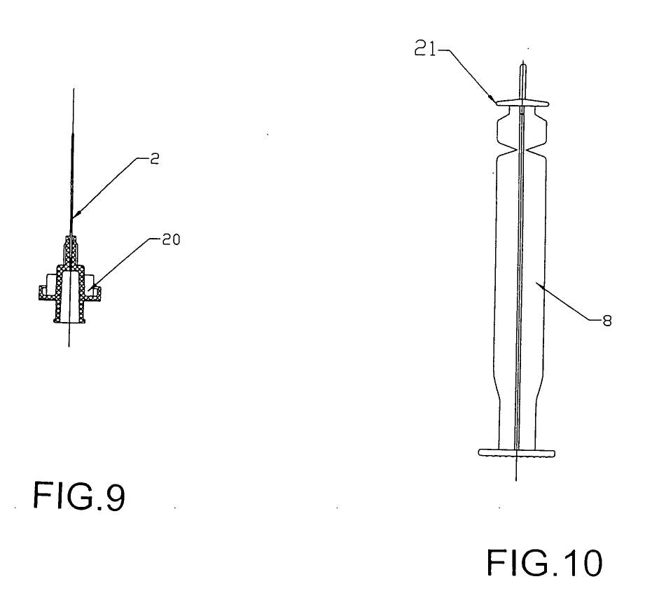

Referring to FIG. 9 of the drawings, an alternative mode of the syringe according to the preferred embodiment of the present invention is illustrated. The alternative mode is similar to the preferred embodiment, except the needle 2 is directly mounted onto the coupling device 5. This arrangement is most suitable for precise injection.

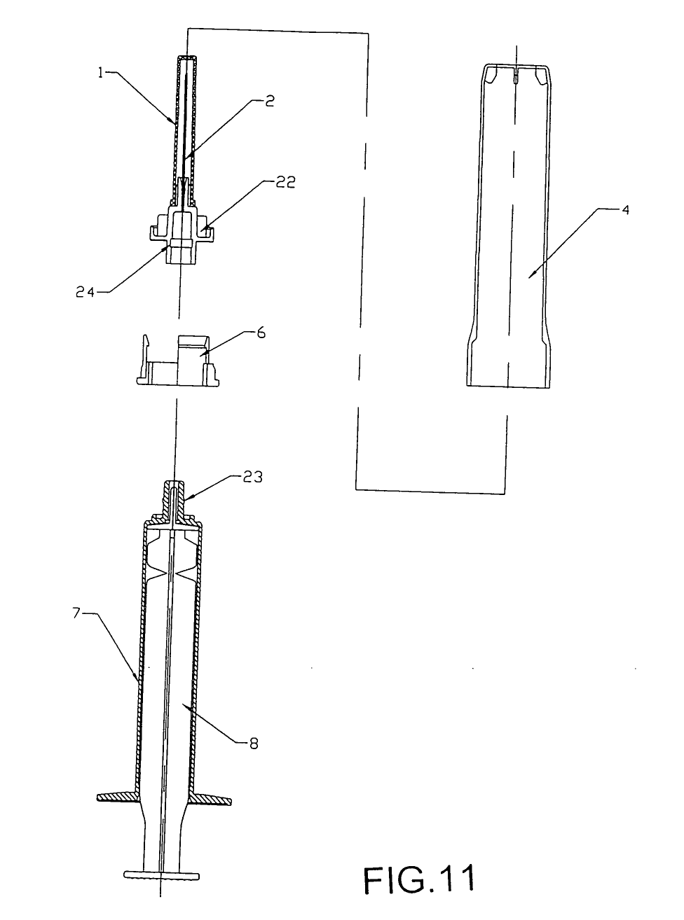

Referring to FIG. 11 of the drawings, a second alternative mode of the preferred embodiment of the present invention is illustrated. The second alternative mode is similar to the preferred embodiment, except the syringe body 7 further comprises an interlocking ring 23 extended from the luer tape 10, wherein the coupling device 5 further has an interlocking slot 24 formed thereon, wherein the interlocking ring 23 is arranged to couple with the interlocking slot 24 so as to enhance the coupling performance between the syringe body 7 and the coupling device 5. Because of this additional feature, the coupling between the syringe body 7 and the coupling device 5 is not affected by transportation or weather conditions so as to produce uniform product quality of the present invention.

What is claimed is:

1. A syringe, comprising a needle cap (1), a needle (2), a needle seat (3), a protective sleeve (4), a coupling device (5), a locking member (6), a syringe body (7), a plunger shaft (8), and a plunger head (9), wherein said needle (2) is affixed to and extended from said needle seat (3), wherein said needle seat is detachably connected to said coupling device (5), which is provided at a top end portion of said protective sleeve (4), while said locking member (6) is received in another end portion of said protective sleeve (4), wherein said locking member is connected with a lower end surface (12) through conventional adhesive agent or welding technology by means of a welding wire (17), wherein said plunger head (9) is provided at a top end portion of said plunger shaft (8), which are movably received within said syringe body, wherein said syringe body, said plunger shaft, and said plunger head are all operatively received in said protective sleeve, wherein said syringe body (7) has a luer taper (10) for connecting with said coupling device through threading mechanism, wherein said coupling device has a circular cross section defining an inner cavity (15), and a plurality of coupling members (16) spacedly extended on said coupling device, wherein said coupling members (16) are arranged to couple with said luer taper (10) of said syringe body, wherein said syringe further comprises a resilient flanges (18) operatively connected with said locking device (6) which interlocks with said needle (2), said needle seat (3) and said coupling device (5) by operation of a sliding action of said flanges (18) in a backward direction.

2. The syringe, as recited in claim 1, wherein said syringe body further comprises an interlocking ring (23) extended from said luer tape (10), wherein said coupling device (5) further has an interlocking slot (24) formed thereon, wherein said interlocking ring (23) is arranged to couple with said interlocking slot (24) so as to enhance a coupling performance between said syringe body and said coupling device.

3. The syringe, as recited in claim 1, wherein said plunger shaft (8) and said plunger head (9) are integrally molded together for receiving in said syringe body, thereby said coupling device (5) and said luer taper (10) of said syringe body (7) are connected in such a manner that a gap is defined between said coupling device (5) and said luer taper (10) of said syringe body (7) through which a residue fluid retained inside said syringe body is restricted for meeting an international standard, wherein said plunger head (9) has a conical shape which is adapted to receive in said luer taper for ejecting a predetermined of fluid from said syringe body.

4. The syringe, as recited in claim 1, wherein said coupling device has a circular cross section defining an inner cavity (15), and a plurality of coupling members (16) spacedly extended on said coupling device, wherein said coupling members (16) are arranged to couple with said luer taper (10) of said syringe body.

5. The syringe, as recited in claim 1, wherein said protective sleeve (4) has an external smooth surface.
